(19) United States
(12) Patent Application Publication
(10) Pub. No.: US 2006/0282072 A1
(43) Pub. Date: Dec. 14, 2006

(54) Electrocautery instrument

(76) Inventor: Paul DesRosier, Tuscan, AZ (US)

Correspondence Address: Ilya Zborovsky, 6 Schoolhouse Way, Dix Hills, NY, 11746, US

(21) Appl. No.: 11/150,645

(22) Filed: Jun. 11, 2005

(51) Int. Cl. A61B 18/14

(52) U.S. Cl. 606/45; 606/49

(57) ABSTRACT

An electrocautery instrument has a cauterizing tool, a hollow, elongated handle having an axis extending in a direction of elongation, a first axial end to which the cauterizing tool is connected and a second opposite axial end, and a structure for illuminating a region around the cauterizing tool and including at least one light bulb which is located inside the first end of the handle and emits light outside of the first end of the handle.

### BACKGROUND OF THE INVENTION

The present invention relates to an electrocautery instrument.

Surgical and electrocautery instruments are known in the art. U.S. Pat. No. 1,449,165 discloses a surgical appliance with illumination, in which the illumination depends on a plug-in connection to a separate instrument providing electric current. This invention does not deliver any RF current. It is an “eye spud” that is no longer used in the treatment of opthamology-related surgeries.

U.S. Pat. No. 2,029,487 discloses an illuminated cautery electrode. It requires AC energy from the RF generator to power the bulb. Therefore it is not possible to provide illumination without electrocautery (RF) delivery. Also, it is not possible here to disable the light mechanism while maintaining RF delivery. The instrument also is not battery powered, and it is specifically stated that the light will be of filament nature, comprised of a lamp bulb, socket, threaded mechanism and shunted electrical relation to the cautery rods.

U.S. Pat. No. 2,196,171 discloses a cautery electrode illuminating device. This is a separate device which is not contained within the cautery pencil. Furthermore, it requires an electrical source other than battery to power the light. It is very complex and is composed of over fifty parts.

U.S. Pat. No. 4,562,838 discloses an electro surgery instrument which however does not have an actual light source located on the cautery pencil. It is a fiber optic cable that would require light to be generated by another separate instrument. This instrument also has to be powered by an AC power source or the light needs to acquire power from the current traveling to the cautery tip.

U.S. Pat. No. 4,657,012 discloses a surgical instrument with incorporated lighting system. It is for a non RF scalpel with a light included. The instrument can not deliver RF energy to cut or to coagulate tissues.

U.S. Pat. No. 4,759,349 discloses a surgical instrument which requires a secondary unit to power the light source within the handle, and there is no battery powered light. It also requires a heat sink to dissipate heat within the pencil by a heat sink mechanism.

U.S. Pat. No. 5,246,440 discloses an electro surgical knife which contains an optical conduit connected to an optical cable, so that there is no light source itself in the instrument. It is also only two-button instrument and it does not have the ability to independently light the operative field. The surgeon or operator can not choose light or no light. These functions are performed by a light source in a completely separate location operated by another person.

U.S. Pat. No. 6,562,032 discloses an electrosurgical instrument with vibration. The light source in this instrument is housed not in the handle of the instrument but instead within an external add-on piece. The light may only be used when current is being applied to the oscillating motor, not by itself.

Finally, U.S. Pat. Nos. DE 253,247 and DE 441,077 discloses electrosurgical instruments in which however the illuminating means are not specifically illustrated and/or explained.

It is believed to be advisable to further improve the existing cautery instruments.

### SUMMARY OF THE INVENTION

Accordingly, it is an object of the present invention to provide an electrocautery instrument which is a further improvement of the existing instruments.

In keeping with these objects and with others which will become apparent hereinafter, one feature of the present invention resides, briefly stated, in an electrocautery instrument which has a cauterizing tool; a hollow, elongated handle having an axis extending in a direction of elongation, a first axial end through which said cauterizing tool extends outwardly and a second opposite axial end; means for delivery RF energy to said cauterizing tool; and means for illuminating a region around said cauterizing tool, said illuminating means including at least one light bulb which is located inside said first end of said handle and emits light outside of said first end of said handle.

When the electrocautery instrument is designed in accordance with the present invention, it has a simple construction, easily powered both by RF energy for operation of the electrocautery tool and for operation of the illuminating means, and very convenient to use for surgical purposes.

In accordance with an important feature of the present invention, a plurality of light bulbs are arranged at the first end of the handle and spaced from one another in a circumferential direction, thus providing a very efficient illumination of the operating region.

In accordance with a further embodiment of the present invention, the first end of the handle has a portion which is inclined to the axis and narrows to the electrocauterizing tool, and the light bulbs are arranged on the inclined surfaces thus directing their light in a concentrated fashion toward the region of surgical operation.

Still another feature of the present invention is that the handle is formed as a substantially one-piece part in which the batteries are accommodated on the second axial end and the bulbs of the illuminating means are accommodated at the first axial end.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view showing an electrocautery instrument in accordance with the present invention;

FIG. 2 is an end view of a first end of the electrocautery instrument in accordance with the present invention, showing a cauterizing tool and illuminating means; and

FIG. 3 is a view showing a connection to a cautery generator.

### DESCRIPTION OF THE PREFERRED EMBODIMENTS

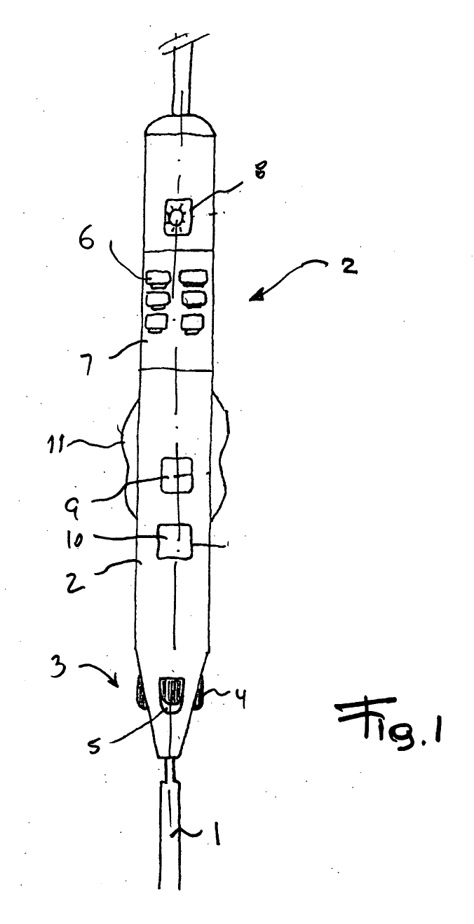

An electrocautery instrument in accordance with the present invention is shown in general in FIG. 1. The electrocautery instrument has a cauterizing tool which is identified with reference numeral 1. The electrocautery instrument further has a handle which is identified with reference numeral 2 and is formed as an elongated handle having an axis extending in a longitudinal direction and two axial ends spaced from one another in the longitudinal direction. The first axial end of the handle is the lower end as considered in FIG. 1.

The electrocautery instrument is further provided with illuminating means which are identified as a whole with reference numeral 3. The illuminating means 3 include at least one light bulb 4 located in an indentation 5 in the first end of the handle. The light bulb can be formed as an LED light bulb. It is believed to be clear that the light bulb is located inside the handle and extends outwardly through the indentation to emit light outwardly.

In accordance with a preferable embodiment of the present invention, the illumination means have a plurality of light bulbs 4 which are spaced from one another in a circumferential direction around the axis, thus providing an efficient illumination of an operating region.

In accordance with still a further feature of the present invention, the first end of the handle in the area of the cauterizing tool 1 has an inclined portion whose outer surface is inclined toward the axis of the instrument and narrows toward the cauterizing tool. The light bulbs are arranged on the inclined portion so that light emitted by the light bulbs also extends at an angle to the axis and is directed to the operating region in a more concentrated manner.

The illuminating means are DC battery operated. For example, an internal lithium ion battery 6 can be accommodated inside a battery compartment 7 in the handle 2. The battery compartment is provided with a removable cover for closing and opening the battery compartment. Otherwise, the handle 2 is formed as a substantially one-piece member.

The electrocautery instrument of the invention is further provided with a light switch identified with reference numeral 8. The light switch can be turned by a surgeon to an ON position and an OFF position so as to activate the illuminating means 3 or to deactivate the same. The light switch is located at the opposite axial end of the handle and is convenient to be operated.

The electrocautery instrument in accordance with the present invention has indicating means for indicating a mode of operation, in particular a coagulating mode or a cutting mode. The indicating means can be formed as two indicators 9 and 10 which for example identify the coagulating mode by blue light and the cutting mode by yellow light. The indicating means can be also powered by the same battery.

In the central portion of the handle between the two opposite axial ends, a grip 11 is provided, which can be ribbed for traction, so that the handle can be comfortably held by a surgeon.

FIG. 2 shows the special inventive arrangement of the light bulbs 4 around the axis of the cautery instrument and also around the cauterizing tool 1, so as to provide a very efficient, and concentrated illumination of the operating region.

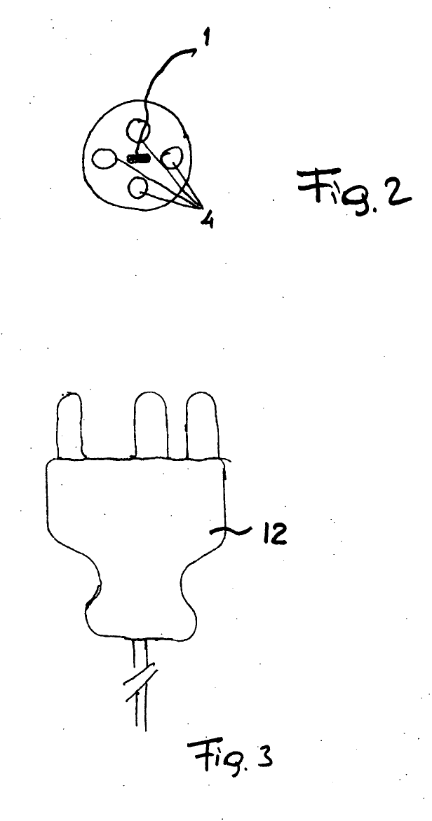

FIG. 3 shows a connection to a cautery generator for supply RF energy. It can be formed as a standard plug 12 which is currently utilized in the corresponding instruments.

It will be understood that each of the elements described above, or two or more together, may also find a useful application in other types of constructions differing from the types described above.

While the invention has been illustrated and described as embodied in an electrocautery instrument, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. An electrocautery instrument, comprising a cauterizing tool; a hollow, elongated handle having an axis extending in a direction of elongation, a first axial end through which said cauterizing tool extends outwardly and a second opposite axial end; means for delivery of RF energy to said cauterizing tool; and means for illuminating a region around said cauterizing tool, said illuminating means including at least one light bulb which is located inside said first end of said handle and emits light outside of said first end of said handle.

2. An electrocautery instrument as defined in claim 1, wherein said illuminating means include a plurality of light bulbs which are located at said first end of said handle and spaced from one another in a circumferential direction around said axis.

3. An electrocautery instrument as defined in claim 1, wherein said first end of said handle has a portion with a surface which is inclined toward said axis so as to narrow toward said electro cauterizing tool, said light bulb being arranged on said inclined surface of said portion of said first end.

4. An electrocautery instrument as defined in claim 1; and further comprising battery means for DC power supply of said illuminating means, said handle having a compartment for said battery means with an opening closeable and openable by a cover, said handle being formed as a one piece element with the exception of said cover.

5. An electrocautery instrument as defined in claim 1; and further comprising switch means operative for turning on and turning off said illuminating means and located in a region of said second end of said handle.

6. An electrocautery instrument as defined in claim 1; and further comprising operating mode indicating means provided on said handle and axially spaced from said illuminating means for indicating a coagulating mode of operation or a cutting mode of operation.

7. An electrocautery instrument as defined in claim 6, wherein said operating mode indicating means are formed as two indicators having different colors.

8. An electrocautery instrument as defined in claim 2, wherein said light bulbs are formed as LED bulbs.
